(19) United States
(12) Patent Application Publication
(10) Pub. No.: US 2008/0003219 A1
(43) Pub. Date: Jan. 3, 2008

(54) DELIVERY OF AN OCULAR AGENT

(75) Inventor: Gholam A. Peyman, Sun City, AZ (US)

Correspondence Address: WOOD, HERRON & EVANS, LLP, 2700 CAREW TOWER, 441 VINE STREET, CINCINNATI, OH, 45202, US

(73) Assignee: MINU, L.L.C., Pittsboro, NC (US)

(21) Appl. No.: 11/425,194

(22) Filed: Jun. 20, 2006

Related U.S. Application Data
(63) Continuation-in-part of application No. 11/348,465, filed on Feb. 6, 2006.
(63) Continuation-in-part of application No. 11/234,970, filed on Sep. 26, 2005.

(51) Int. Cl. A61K 31/506; A61K 31/56; A61K 31/7105; A61K 39/395; A61P 29/00

(52) U.S. Cl. 424/133.1; 424/145.1; 514/171; 514/252.18; 514/252.19; 514/44

(57) ABSTRACT

A method to ameliorate inflammatory effects by providing an anti-platelet derived growth factor (anti-PDGF) agent. In one embodiment the agent is imatinib. In another embodiment the agent is dasatinib. The method reduces the undesirable effects of post-surgical scarring, such as restenosis, because of the anti-inflammatory effect of the anti-PDGF agent. The method may be used in patients undergoing any type of surgery, such as cardiac surgery, ocular surgery, vascular surgery, etc.

This application is a continuation-in-part of pending U.S. patent application Ser. No. 11/348,465, filed on Feb. 6, 2006, which is a continuation-in-part of U.S. application Ser. No. 11/234,970, filed on Sep. 26, 2005, each expressly incorporated by reference herein in its entirety.

This application is related to commonly assigned, copending applications, Ser. Nos. 11/348,151 and 11/348,017, each filed Feb. 6, 2006 and entitled DEVICE FOR DELIVERY OF AN AGENT TO THE EYE AND OTHER SITES, and DELIVERY OF AN AGENT TO AMELIORATE INFLAMMATION, respectively each naming Peyman as the inventor, each of which is expressly incorporated by reference herein in its entirety.

### BRIEF DESCRIPTION OF THE DRAWINGS

FIG. 1 is a schematic cross-sectional view of a mammalian eye.

FIG. 2 is an enlarged diagrammatic illustration of the circled area in FIG. 1

In one embodiment, a method is disclosed for controlling, reducing, or preventing inflammation, an anti-inflammatory response, and/or effects of an anti-inflammatory response, encompassed generally as ameliorating inflammation. The method provides to a patient an anti-vascular endothelial growth factor (VEGF) agent to ameliorate inflammation. Anti-VEGF agents include but are not limited to bevacizumab (rhuMab VEGF, Avastin®, Genentech, South San Francisco Calif.), ranibizumab (rhuFAb V2, Lucentis®, Genentech), pegaptanib (Macugen®, Eyetech Pharmaceuticals, New York N.Y.), sunitinib maleate (Sutent®, Pfizer, Groton Conn.), TNP470, integrin av antagonists, 2-methoxyestradiol, paclitaxel, and P38 mitogen activated protein kinase inhibitors. Anti-VEGF siRNA (short double-stranded RNA to trigger RNA interference and thereby impair VEGF synthesis) may also be used as an anti-VEGF agent.

In one embodiment, the anti-VEGF agent is bevacizumab, administered either alone or with one or more agent(s) known to one skilled in the art under the classification of anti-inflammatory agents. These include, but are not limited to, steroids, anti-prostaglandins, matrix metalloproteinase inhibitors, non-steroidal anti-inflammatory drugs (NSAIDS), macrolides, anti-proliferative agents, anti-cancer agents, etc. In one embodiment, the method ameliorates inflammation using the anti-VEGF agent such as bevacizumab alone. In another embodiment, the method ameliorates inflammation using the anti-VEGF agent such as bevacizumab to supplement known anti-inflammatory agents. In both embodiments, the method ameliorates inflammation at any stage, even early stage inflammation before occurrence of an angiogenic component. The method controls inflammation, and counteracts the action of angiogenic agents such as VEGF on the permeability of a vessel wall, thereby reducing or preventing the resulting tissue damage due to fluid leakage from the vessel (extravasation). The method is applicable to any tissue or site in the body, and to any cause of inflammation such as immune disease including autoimmune disease, viral and/or bacterial infection, trauma including surgical trauma, etc. In one embodiment, the method controls, reduces, or prevents tissue damage in the brain. In one embodiment, the method controls, reduces, or prevents tissue damage in the eye.

Inflammation is a localized, protective response of vascularized tissue to sub-lethal tissue injury or destruction. The response functions to destroy, dilute, or sequester both the injurious agent and the injured tissue. Inflammation can be classified according to duration as either acute or chronic. In the acute form of an inflammatory response, classical signs are pain, heat, redness, swelling, and loss of function. Histologically, there are a complex series of events including dilatation of arterioles, capillaries and venules, with increased permeability and blood flow, exudation of fluids including plasma proteins, and leukocyte migration and accumulation at the site of injury. This reaction may trigger a systemic response such as fever, leukocytosis, protein catabolism, and altered hepatic synthesis of plasma proteins such as C-reactive protein. Chronic inflammation is characterized by macrophage and lymphocyte infiltration into the affected and surrounding tissue.

Inflammation is a homeostatic response to tissue damage by a range of stimuli, including infection and trauma. For example, an inflammatory response helps to destroy or inactivate invading pathogens. In cases of autoimmune diseases such as rheumatoid arthritis, etc., inflammation is a response against self. The inflammatory process removes waste and debris and restores normal function, either through resolution or repair. Tissue structure is normal after resolution, whereas repair leads to a functional, but morphologically altered, organ. In acute inflammation, tissue damage is followed by resolution or healing by scar formation, whereas in chronic inflammation, damage and repair continue concurrently. The initial inflammatory response is usually acute, and may or may not evolve into chronic inflammation. However, chronic inflammation is not always preceded by an acute phase. Although usually beneficial to the organism, inflammation itself may lead to tissue damage, resulting in escalation of chronic inflammation. Inflammation underlies the pathology of virtually all rheumatologic diseases. The severity of disorders, such as arthritis, is classified according to the degree of inflammation and its destructive effects.

Anti-VEGF agents affect the process of angiogenesis, which is the growth of new blood vessels from pre-existing vasculature. It is a fundamental process required for embryogenesis, growth, tissue repair after injury, and the female reproductive cycle. It also contributes to the pathology of conditions such as cancer, age related macular degeneration, psoriasis, diabetic retinopathy, and chronic inflammatory diseases in joints or lungs. Angiogenesis is stimulated when hypoxic, diseased, or injured tissues produce and release angiogenic promoters such as VEGF, platelet derived growth factor (PDGF), or fibroblast growth factor (FGF)-1. These angiogenic factors stimulate the migration and proliferation of endothelial cells in existing vessels and, subsequently, the formation of capillary tubes and the recruitment of other cell types to generate and stabilize new blood vessels.

Angiogenic factors may be pro-inflammatory factors. Relatively minor irritation of internal tissues, such as occurs during surgery, does not lead to neovascularization, but encourages tissue adhesion and scarring. Agents that inhibit angiogenesis such as the previously disclosed TNP470, integrin av antagonists, 2-methoxyestradiol, paclitaxel, P38 mitogen activated protein kinase inhibitors, anti-VEGF siRNA, and sunitinib maleate (Sutent®/SU11248) or anti-platelet derived growth factor agents such as imatinib (Gleevec®, Novartis AG, Basel Switzerland) or dasatinib (Bristol-Myers Squibb-354825) may inhibit synovitis, uveitis, iritis, retinal vasculitis, optic nerve neuritis, papillitis, retinitis proliferance in diabetes, etc. Expression of adhesion molecules such as integrin avb3 and e-selectin are upregulated in new vessels, and new vessels appear sensitive to inflammogens. The angiogenic factor FGF-1 enhances antigen-induced synovitis in rabbits, but is not pro-inflammatory when administered alone. However, angiogenesis occurs in the absence of inflammation such as during embryonic growth and in the female reproductive cycle. Thus, inflammation and angiogenesis can occur independently and administration of anti-VEGF agents such as bevacizumab, either alone or to supplement known anti-inflammatory agents, ameliorates both inflammation without an angiogenic component (earlier stage inflammation), and inflammation that has progressed to an angiogenic component (later stage inflammation). Coexistence of inflammation and angiogenesis may lead to more severe, damaging, and persistent inflammation.

Angiogenesis enhances tumor growth, and anti-angiogenic agents are used clinically. Mechanisms by which new vessels enhance tumor growth include providing metabolic requirements of the tumor, generating growth factors by vascular cells, and inhibiting apoptosis. Inhibiting the function of growth factors such as VEGF can reduce or prevent pathological angiogenesis in tumors.

Angiogenesis may also contribute to thickening of airways in asthma and of lung parenchyma in pulmonary fibrosis, and to growth of sarcoid granulomas. Growth of granulation tissue into airspaces also may be angiogenesis-dependent in bronchi after lung transplant and in alveoli after acute lung injury or in other forms of pulmonary fibrosis. Angiogenesis may also contribute to growth of the synovial pannus in rheumatoid arthritis. Interposition of expanded, innervated synovium between articulating surfaces may contribute to pain on movement. In each of these situations, the expanded tissue may impair function.

The new blood vessels that result from angiogenesis have incomplete walls and are particularly susceptible to disruption and fluid extravasation. This has been proposed as a cause of pulmonary hemorrhage in inflammatory lung disease. Hemosiderin deposits and extravasated erythrocytes are commonly present in inflammatory synovitis, although the contribution of angiogenesis to synovial microhemorrhage is unknown, and its contribution to synovial inflammation remains unclear. The inflammatory potential is evident, however, in patients with hemophilia.

Angiogenesis occurs as an orderly series of events, beginning with production and release of angiogenic growth factors (proteins) that diffuse into nearby tissues. The angiogenic growth factors bind to specific receptors located on the endothelial cells of nearby preexisting blood vessels. Once growth factors bind to their receptors, the endothelial cells are activated and begin to produce enzymes and other molecules that dissolve tiny holes in the sheath-like basement membrane that surrounds existing blood vessels. The endothelial cells begin to divide and proliferate, and they migrate through the holes of the existing vessel towards the diseased tissue or tumor. Specialized adhesion molecules or integrins (avb3, avb5) help to pull the new blood vessels forward. Additional enzymes, termed matrix metalloproteinases (MMP), are produced and dissolve the tissue in front of the sprouting vessel tip in order to accommodate it. As the vessel extends, the tissue is remolded around the vessel. Sprouting endothelial cells roll up to form a blood vessel tube and individual blood vessel tubes connect to form blood vessel loops that can circulate blood. The newly formed blood vessel tubes are stabilized by smooth muscle cells, pericytes, fibroblasts, and glial cells that provide structural support, permitting blood flow to begin.

VEGF is a specific angiogenesis growth factor that binds to receptors on blood vessels and stimulates the formation of new blood vessels. VEGF is a potent inducer of both endothelial cell proliferation and migration, and its biologic activities are largely specific for endothelial and vascular smooth muscle cells. Unlike basic fibroblast growth factor (bFGF), high levels of VEGF are not present in early surgical wounds. Rather, VEGF levels peak seven days after the wound is created, at which point VEGF appears to be a major stimulus for sustained induction of blood vessel growth and high levels of PDGF have been shown. There are abundant sources of VEGF in wounds. Many cell types produce VEGF, including keratinocytes, macrophages, fibroblasts, and endothelial cells. Thus, there is massive VEGF secretion, particularly in the setting of hypoxia, which is often observed in wounds.

Anti-VEGF agents inhibit the action of VEGF. As one example of an anti-VEGF agent, bevacizumab is a recombinant humanized monoclonal IgG1 antibody that binds to and inhibits the biologic activity of human VEGF in in vitro and in vivo assay systems by preventing binding of VEGF with its receptor on the surface of vascular endothelial cells, thus preventing endothelial cell proliferation and new vessel formation. Bevacizumab contains human framework regions and the complementarity-determining regions of a murine antibody that binds to VEGF; it has a molecular weight of about 149 kilodaltons. Bevacizumab, by binding to VEGF, blocks VEGF from binding to receptors and thus blocks angiogenesis. Bevacizumab is typically administered by intravenous infusion, diluted in 0.9% sodium chloride for injection from a 25 mg/ml preparation.

Ranibizumab is a derivative of the full-length antibody bevacizumab (Fab fragment), and is further modified to increase its affinity for VEGF. Both bevacizumab and ranibizumab bind all biologically active isoforms and proteolytic fragments of VEGF, but there are differences. Monovalent binding of a Fab fragment such as ranibizumab to its target antigen would not force the target to dimerize, and hence is useful to manipulate cell receptor function, but its effective antigen binding capacity is lower than its full antibody counterpart. However, VEGF, which is the desired target, is a soluble factor and not a cellular receptor. Therefore, the increased effective binding by the full length antibody bevacizumab enhances inhibition of the VEGF signal and thus provides an enhanced anti-angiogenic effect. Bevacizumab has also been “humanized” to decrease any antigenic effect it may have on the patient, and bevacizumab has a higher molecular weight; this full-length antibody likely will not penetrate the retina to the same extent as the lower molecular weight fragment ranibizumab. However, the increased size of bevacizumab may decrease its clearance rate from the site of action.

Sunitinib maleate (Sutent®) is an orally bioavailable indolinone with potential antineoplastic activity. It blocks the tyrosine kinase activities of vascular endothelial growth factor receptor 2 (VEGFR2), platelet-derived growth factor receptor b (PDGFRb), and c-kit, thereby inhibiting angiogenesis and cell proliferation. This agent also inhibits the phosphorylation of Fms-related tyrosine kinase 3 (FLT3), another receptor tyrosine kinase expressed by some leukemic cells. (NC104). A systemic dose for cancer treatment is between 12.5 mg/day to 50 mg/day.

Imatinib (Gleevec®) is also a potent anti-PDGF compound. It is used clinically as an antineoplastic against chronic myeloid leukemia (CML). Although most CML patients respond well to imatinib, about 10% who initially respond subsequently develop drug resistance, which may be due to mutations in bcr-abl tyrosine kinase. Dasatinib (Sprycel® Bristol Myers Squibb, BMS-354825) is another orally bioavailable small-molecule tyrosine kinase inhibitor with strong PDGFR effects. Dasatinib is more potent (300 to 1000 times) than imatinib for inhibiting bcr-abl tyrosin kinase, likely because it can more tightly bond with the bcr-abl tyrosine kinase target and thus overcome imatinib resistance caused by the bcr-abl tyrosine kinase mutation. Dasatinib is also an Src kinase inhibitor as well as an abl inhibitor. Another agent that has similar properties to Dasatinib is AMN107 (Novartis, Basel Switzerland). As anti-PDGF agents, imatinib, dasatinib, and AMN107 have anti-inflammatory action.

Among the available anti-inflammatory agents, many have a target of action to block or ameliorate the actions of pro-inflammatory signals, such as histamine and cytokines. Although this provides some relief from the harmful effects of inflammation, it does not address the cause of the problem. Leukocytes and macrophages, which release pro-inflammatory factors into affected areas, are allowed access to the inflamed tissue following new blood vessel formation.

In one embodiment, the inventive method administers one or a combination of anti-VEGF agent(s) such as bevacizumab, ranibizumab, pegaptanib, sunitinib maleate, etc., and/or anti-PDGF agents such as imatinib, dasatinib, etc. as the sole agent(s) to ameliorate inflammation, and thus to control, reduce or prevent an inflammatory response or ameliorate the effects of an inflammatory response. In one embodiment, bevacizumab is used to enhance reabsorption of inflammatory exudates. Decreasing the level of exudates in the eye reduces the inflammatory process and the ensuing hyperpermeable state that occurs with allergies, infection, responses to ocular photodynamic therapy (PDT) and laser treatments, after ocular surgery or trauma, etc. In one embodiment, the anti-VEGF agent is administered to ameliorate an inflammatory process without an angiogenic component. Many inflammatory processes, such as early stage inflammation, are not associated with the formation of new blood vessels. Examples include, but are not limited to, inflammatory diseases of the central nervous system (brain and spinal cord) such as abscess, meningitis, encephalitis, vasculitis, and conditions resulting in cerebral edema; inflammatory diseases of the eye (uveitis, subsequently discussed), macular edema, and others known to one skilled in the art.

In one embodiment, the anti-VEGF agent is administered to ameliorate the scarring and adhesions that are a part of the inflammatory process. Adhesions are bands of scar tissue that bind two internal body surfaces. They are an inflammatory response to tissue damage, and occur as a normal part of any healing process. As one example, adhesions frequently occur during the post-surgical healing process during which tissues have experienced mechanical trauma. However, adverse effects can occur when internal surfaces bind, and adhesions may persist even after the original trauma has healed. Surgery to repair adhesions itself results in recurrent or additional adhesions. The presence of adhesions may also complicate surgical procedures, for example, ocular conjunctival adhesions may complicate subsequent glaucoma surgery.

Adhesions can occur following any type of trauma or surgery, including but not limited to ocular surgery. Examples of ocular surgery that may result in adhesions include glaucoma filtration operations (i.e., iridencleisis and trephination, pressure control valves), extraocular muscle surgery, diathermy or scleral buckling surgery for retinal detachment, and vitreous surgery. Examples of ocular trauma include penetrating ocular injuries, intraocular foreign body, procedures such as PDT, scatter laser threshold coagulation, refractive surgery, and blunt trauma.

In one embodiment, imatinib, dasatinib, and/or AMN107 may be used as an anti-inflammatory agent to minimize, reduce, or prevent post-surgical scar tissue formation. Scar formation occurs as a normal post-surgical process. However, scar formation following cardiac and/or vascular surgery, including grafts, can result in narrowing of a vessel lumen, which can reduce blood flow to an organ. Scar formation following ocular surgery can result in decreased visual acuity. In one embodiment, these agents may be administered, singly or in combination, in post-stent therapy to reduce restenosis. Restenosis is a re-narrowing or blockage of an artery at a site of treatment (e.g., angioplasty, stent procedure). This narrowing or blockage reduces blood flow to the heart. Cells naturally form around the stent but, in some cases, cellular overgrowth occurs similar to scar tissue formation that occurs during an inflammatory process. Use of imatinib, dasatinib, and/or AMN107 reduces or prevents such processes and thus can reduce or minimize restenosis. In another embodiment, these agents may be administered, singly or in combination, after ocular surgery to reduce ocular scar formation. As one example, these agents may be administered topically, by subconjunctival injection, by intravitreal injection, or by other ocular routes, or may be administered systemically, using methods known to one skilled in the art, to a patient following ocular surgery. In one embodiment, the administered dose ranges from about 0.1 μg/ml to about 5 mg/ml. In another embodiment, the administered dose ranges from about 1 μg/ml to about 100 μg/ml.

In one embodiment, anti-VEGF agents ameliorate disorders with both a vascular proliferative component and a scarring component. As one example, the invention may be used in patients with the ocular disease pterygia. In these patients, fibrovascular proliferation results in scarring of the conjunctiva. An elevated, superficial, external ocular mass, termed a pterygium, forms and extends onto the corneal surface. Patients may experience symptoms of inflammation (e.g., redness, swelling, itching, irritation) and blurred vision. The mass itself may become inflamed, resulting in redness and ocular irritation. Left untreated, pterygia can distort the corneal topography, obscure the optical center of the cornea, and result in altered vision.

The process whereby scar tissue forms (scarring) can occur without new blood vessels being formed (neovascularization). However, the neovascularization process always results in scarring because of the cell proliferation that occurs with the formation of new vessels also results in the proliferation of fibroblasts, glial cells, etc. that result in scar tissue formation. The inventive method may be used to ameliorate the scarring process.

In one embodiment, the anti-VEGF agent is administered to ameliorate inflammation of uveal tissues (uveitis, an inflammation of tissues in the middle layer of the eye, mainly the iris (iritis) and the ciliary body). Ocular inflammation may be associated with underlying systemic disease or autoimmunity, or may occur as a direct result of ocular trauma or infectious agents (bacterial, viral, fungal, etc.). Inflammatory reactions in adjacent tissues, e.g., keratitis, can induce a secondary uveitis. There are both acute and chronic forms of uveitis. The chronic form is frequently associated with many systemic disorders and most likely occurs due to immunopathological mechanisms.

Uveitis presents with ocular pain, photophobia and hyperlacrimation, with decreased visual acuity ranging from mild blur to significant vision loss. Hallmark signs of anterior uveitis are cells and flare in the anterior chamber. If the anterior chamber reaction is significant, small gray to brown endothelial deposits known as keratic precipitates may arise, leading to endothelial cell dysfunction and corneal edema. There may be adhesions to the lens capsule (posterior synechia) or the peripheral cornea (anterior synechia). Granulomatous nodules may appear on the surface of the iris stroma. Intraocular pressure is initially reduced due to secretory hypotony of the ciliary body but, as the reaction persists, inflammatory by-products may accumulate in the trabeculum. If this debris builds significantly, and if the ciliary body resumes its normal secretory output, the pressure may rise sharply, resulting in a secondary uveitic glaucoma.

One skilled in the art will appreciate that scarring and adhesions in areas of the body other than the eye may be treated with the inventive method. Examples include adhesions associated with cardiac surgery (e.g., angioplasty, stent placement, grafts, adhesions in the pericardial space), pulmonary surgery (e.g., in the periplural space), abdominal surgery (e.g., appendectomy, gastric bypass surgery), gynecological surgery (e.g., episiotomy, Caesarean section, hysterectomy), any type of laparoscopy or laparotomy surgery, reconstructive surgery (cosmetic or therapeutic), organ removal (partial or complete), etc.

In another embodiment, the inventive method administers an anti-inflammatory agent simultaneously or concomitantly with an anti-VEGF agent such as bevacizumab and thus controls, reduces, or prevents an inflammatory response. Other anti-VEGF agents such as Lucentis®, Macugen®, Sutent®, geldanamycin, etc. may be included.

The method may be used for any tissue including, but not limited to, eye (e.g., to ameliorate conjunctivitis (inflammation of the conjunctivae, the mucous membranes covering the sclera and inner eyelid), that may be associated with bacterial, viral, or Chlamydia infections, allergies, or susceptibility to irritants such as chemicals, smoke, etc., lung (e.g., to ameliorate interstitial lung disease, inflammation of the interstitium (tissue between the air sacs in the lung)), bone (e.g., to ameliorate synovitis, inflammation of the synovium (the membranes lining joints) that may be associated with arthritis), brain (e.g., to ameliorate encephalitis (inflammation of brain tissue and/or membranes)), and muscle (e.g., to ameliorate myopathies (inflammation of muscles, such as muscles near a joint)). The method may be used on patients at risk for developing inflammation. The method may be used on patients with inflammation and/or inflammatory processes from any cause, including but not limited to autoimmune diseases, diseases with an immune component, ischemic diseases, diabetes, age related macular degeneration, retinitis pigmentosa, infectious diseases, allergen-induced inflammation, other degenerative diseases, etc.

In the embodiment where the anti-VEGF agent(s) is administered with an anti-inflammatory agent, an effective amount of the anti-inflammatory agent is administered to a patient at a standard dose known to one skilled in the art. As one example, prednisone is administered for a systemic dose in the range between about 5 mg to about 100 mg daily. As another example, Solu-medrol® is administered intravenously in a single dose of about 1 mg. Other anti-inflammatory agents, possible routes of administration, doses, etc. are known to one skilled in the art. The agent may be administered by any route including enteral and parenteral route, for example, intravenously, orally, ocularly, etc. One skilled in the art will appreciate that the route of administration may vary due to factors such as agent solubility, patient needs, dose required, etc. The anti-inflammatory agent may be a fast-acting anti-inflammatory agent, a slow acting anti-inflammatory agent, or both a fast-acting and a slow-acting anti-inflammatory agent. The anti-inflammatory agent may be formulated for delayed and/or extended release to provide effects over a longer period of time.

Examples of anti-inflammatory agents recognized by one skilled in the art include, but are not limited to, the following: colchicine; a steroid such as triamcinolone (Aristocort®; Kenalog®), anecortave acetate (Alcon), betamethasone (Celestone®), budesonide cortisone, dexamethasone (Decadron-LA®; Decadron® phosphate; Maxidex® and Tobradex® (Alcon)), hydrocortisone methylprednisolone (Depo-Medrol®, Solu-Medrol®), prednisolone (prednisolone acetate, e.g., Pred Forte® (Allergan), Econopred and Econopred Plus® (Alcon), AK-Tate® (Akorn), Pred Mild® (Allergan), prednisone sodium phosphate (Inflamase Mild and Inflamase Forte® (Ciba), Metreton® (Schering), AK-Pred® (Akorn)), fluorometholone (fluorometholone acetate (Flarex® (Alcon), Eflone®), fluorometholone alcohol (FML® and FML-Mild®, (Allergan), FluorOP®), rimexolone (Vexol® (Alcon)), medrysone alcohol (HMS® (Allergan)), lotoprednol etabonate (Lotemax® and Alrex® (Bausch & Lomb), and 11-desoxcortisol; an anti-prostaglandin such as indomethacin; ketorolac tromethamine; ((+)-5-benzoyl-2,3-dihydro-1H-pyrrolizine-1-carboxylic acid, a compound with 2-amino-2-(hydroxymethyl)-1,3-propanediol (1:1) (Acular® Allegan), Ocufen® (flurbiprofen sodium 0.03%), meclofenamate, fluorbiprofen, and the pyrrolo-pyrrole group of non-steroidal anti-inflammatory drugs; a macrolide such as sirolimus (rapamycin), pimocrolous, tacrolimus (FK506), cyclosporine (Arrestase), everolimus 40-0-(2-hydroxymethylenrapamycin), ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, biolimus, ABT-578 (methylrapamycin), and derivatives of rapamycin such as temsirolimus (CCI-779, Wyeth) and AP23573 (Ariad); a non-steroidal anti-inflammatory drug such as derivatives of acetic acid (e.g. diclofenac and ketorolac (Toradol®, Voltaren®, Voltaren-XR®, Cataflam®)), salicylate (e.g., aspirin, Ecotrin®), proprionic acid (e.g., ibuprofen (Advil , Motrin®, Medipren®, Nuprin®)), acetaminophen (Tylenol®), aniline (e.g., aminophenolacetaminophen, pyrazole (e.g., phenylbutazone), N-arylanthranilic acid (fenamates) (e.g., meclofenamate), indole (e.g., indomethacin (Indocin®, Indocin-SR®)), oxicam (e.g., piroxicam (Feldene®)), pyrrol-pyrrole group (e.g., Acular®), antiplatelet medications, choline magnesium salicylate (Trilisate®), cox-2 inhibitors (meloxicam (Mobic®)), diflunisal (Dolobid®), etodolac (Lodine®), fenoprofen (Nalfon®), flurbiprofen (Ansaid®), ketoprofen (Orudis®, Oruvail®), meclofenamate (Meclomen®), nabumetone (Relafen®), naproxen (Naprosyn®, Naprelan®, Anaprox®, Aleve®), oxaprozin (Daypro®), phenylbutazone (Butazolidine®), salsalate (Disalcid®, Salflex®), tolmetin (Tolectin®), valdecoxib (Bextra®), sulindac (Clinoril®), and flurbiprofin sodium (Ocufen®), an MMP inhibitor such as doxycycline, TIMP-1, TIMP-2, TIMP-3, TIMP-4; MMP1, MMP2, MMP3, Batimastat (BB-94), TAPI-2,10-phenanthroline, and marimastat. The composition may contain anti-PDGF compound(s) such as imatinib mesylate (Gleevec®), sunitinib malate (Sutent®) which has anti-PDGF activity in addition to anti-VEGF activity, and/or anti-leukotriene(s) such as genleuton, montelukast, cinalukast, zafirlukast, pranlukast, zileuton, BAYX1005, LY171883, and MK-571 to account for the involvement of factors besides VEGF in neovascularization. The composition may additionally contain other agents including, but not limited to, transforming growth factor β (TGFβ), interleukin-10 (IL-10), aspirin, a vitamin, and/or an antineoplastic agent.

An effective amount of anti-VEGF agent, either as the sole active agent, or with one or more other non-antiinflammatory agents as previously described, is administered. Administration of either agent may be by any route, and the agents may be administered by the same route or by different routes, including enteral, parental, and ocular routes such as intravitreal injection, subconjunctival injection, retrobulbar injection, topical, etc. As one example, the anti-VEGF agent (bevacizumab, sunitinib, etc.) may be topically administered to intact or compromised eyes, skin, mucous membranes, etc. to reduce scarring after trauma, surgery, radiation, burns, wounds, etc. As another example, it may be locally administered to a site in a surgical field to ameliorate inflammation (e.g., adhesions, scarring, effusions) of pleura, epicardium, etc. after thoracic, cardiac, abdominal, etc. surgery. As another example, it may be administered intrathecally (brain, spinal cord, etc.). As another example, it may be administered by inhalation, for example, to ameliorate inflammation in the respiratory tract (nose, trachea, bronchi, lungs, etc.). As another example, it may be instilled in a body cavity (ventricles, sinuses, bladder, etc.). As another example, sunitinib may be administered systemically (e.g., a single dose/week for one month, then monthly reevaluation of need) or topically (e.g., from about 10 ng/ml to about 100 ng/ml), or intraocularly (e.g., from about 7 ng/ml to about 20 μg/ml). In one embodiment, the administered dose of bevacizumab is less than about 5 mg/0.1 ml. In another embodiment, the administered dose of bevacizumab ranges from 0.1 mg/ml to about 50 mg/ml. In another embodiment, the dose of bevacizumab administered systemically ranges from about 0.05 mg/ml to about 5 mg/ml. In one embodiment, the dose of bevacizumab administered intraocularly (e.g., intravitreally) is about 0.005 mg/0.1 ml to about 5 mg/0.1 ml. In one embodiment, the dose of bevacizumab administered topically to the eye is up to 5 mg/ml, and in another embodiment it may be higher. While these doses recite bevacizumab, one skilled in the art will appreciate that they may be used with other anti-VEGF agents, and that doses for a specific agent may be determined empirically, by patient disease severity, other patient variables, etc.

Solutions may be prepared using a physiological saline solution as a vehicle. The pH of an ophthalmic solution may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulations may also contain pharmaceutically acceptable excipients known to one skilled in the art such as preservatives, stabilizers, surfactants, chelating agents, antioxidants such a vitamin C, etc. Preservatives include, but are not limited to, benzalkonium chloride, chlorobutanol, thimerosal, phenylmercuric acetate and phenylmercuric nitrate. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. In one embodiment, bevacizumab and/or other anti-VEGF agent(s) may be administered via a controlled release system (i.e., delayed release formulations and/or extended release formulations) such as polylactic or polyglycolic acid, silicone, hema, and/or polycaprolactone microspheres, microcapsules, microparticles, nanospheres, nanocapsules, nanoparticles, etc. A slow release system may release about 10 ng anti-VEGF agent/day to about 50 ng anti-VEGF agent/day for an extended period.

In various embodiments, the compositions may contain other agents. The indications, effective doses, formulations, contraindications, vendors, etc. of these are available or are known to one skilled in the art. It will be appreciated that the agents include pharmaceutically acceptable salts and derivatives.

Administration of an anti-VEGF agent such as bevacizumab, and optionally other agents such as an anti-PDGF agent, another anti-VEGF agent, etc., may supplement or replace PDT and hence avoid the retinal damage frequently associated with PDT. PDT is frequently used to reduce or prevent damage from leaky vessels associated with age related macular degeneration and other diseases. A series of PDT treatments is often performed with a cumulative effect that, over time, results in retinal damage which in some cases may be severe. The present invention may obviate the need for PDT thus eliminating its associated damage.

In one embodiment, sunitinib maleate (Sutent®) may be used to ameliorate (e.g., reduce, prevent, slow, etc.) age related macular degeneration (AMD), either alone or in combination with PDT or laser coagulation therapy (e.g., scatter threshold laser coagulation, etc.) (such therapies are described in U.S. Pat. No. 6,942,655, which is expressly incorporated by reference herein in its entirety). Sunitinib maleate, alone or in combination with such therapies, is administered to improve vision, maintain vision, or reduce loss of visual acuity in a patient having or at risk for developing AMD. By reducing, slowing, or preventing its onset or progression, it thus reduces effects of ARMD.

In one embodiment, a substantially non-toxic dose of sunitinib maleate is intraocularly administered. Because patients with early stage AMD may receive PDT, there may be cumulative inflammatory effects. Inflammation may result from an immune disease or reaction, including autoimmune diseases, or the presence of a foreign body or organism in the eye. It may be due to macular edema from any cause.

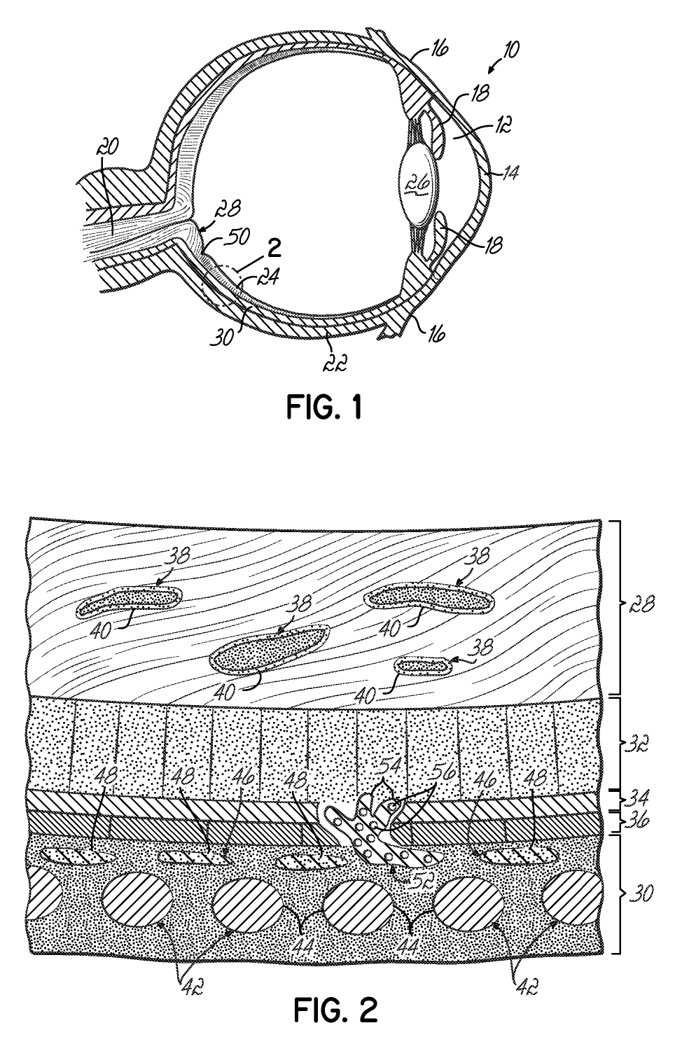

In one embodiment, sunitinib maleate is administered as the sole agent. It may be administered orally at a dose ranging between about 12.5 mg/day to about 50 mg/day. It may be administered topically at a dose ranging between about 10 ng/ml to about 100 ng/ml. It may be administered intraocularly at a dose between about 7 ng/ml to about 20 μg/ml. In one embodiment, it is administered intraocularly using, as shown in FIG. 1, a device 5 placed in eye 10. The device is completely described in co-pending application DEVICE FOR DELIVERY OF AN AGENT TO THE EYE AND OTHER SITES previously incorporated by reference herein. The agent may be formulated as a liquid, suspension (e.g., small particulates suspended in a liquid), etc. It will be appreciated that other formulations, including but not limited to emulsions, microspheres, liposomes, nanoparticles, nanospheres, etc. may also be delivered by the device. It may be administered by a controlled release system, as previously described, formulated as known by one skilled in the art, to release about 10 ng/day to about 50 ng/day over several years. The dose may be administered in any convenient volume (e.g. from about 0.1 ml to about 0.5 ml). One skilled in the art will appreciate that doses for a specific patient may be determined empirically, by disease severity, the presence of other pathologies, other patient variables such as age and gender, etc.

The individual using the inventive method may be at risk for developing AMD, may present with one or more symptoms of AMD, and/or may be already undergoing therapy for AMD using other therapies, either singly or in combination. The method delays the onset or severity of the symptoms of AMD, improving visual acuity and/or preventing further vision loss, and/or reducing the need for retreatments. The combination of anti-VEGF agents with other anti-inflammatory agents results in collective or synergistic action to reduce or halt disease progression.

In one embodiment, and without being bound or limited by a specific theory, it is believed that the method achieves a synergistic effect when ocular phototherapy, for example, PDT, scatter threshold laser coagulation, other types of laser therapy, etc., is administered substantially in conjunction with sunitinib maleate. The therapies damage the existing lesion of nascent vessels, and reduce the recurrence and slow the progression of additional new vessels. The therapies may be administered in any sequence, that is, sunitinib maleate may be administered before or after PDT, etc. or they may be administered essentially simultaneously, as discussed in more detail below. In one embodiment, sunitinib maleate may be administered prior to laser treatment. In this embodiment, sunitinib maleate treatment will decrease existing subretinal exudates, rendering subsequent laser treatment more effective. In this embodiment, sunitinib maleate treatment will reduce subsequent hyperpermeability that results because of the release of VEGF as a consequence of laser procedures.

AMD is a pathological, progressive age-related degeneration in the macula lutea 24 of the retina 28 (FIG. 1 is a schematic cross-sectional view of a mammalian eye 10 showing the anterior chamber 12, cornea 14, conjunctiva 16, iris 18, optic nerve 20, sclera 22, macula lutea 24, lens 26, retina 28 and choroid 30; the previously disclosed device 5 is also shown). AMD is the most common cause of legal blindness among individuals over the age of 60, with an incidence ranging from 11% to 18.5% in individuals over the age of 85. In the United States, AMD affects roughly 3.6 million individuals, with more than 200,000 new cases developing annually.

FIG. 2 is an enlarged diagrammatic illustration of the circled area 2 in FIG. 1 showing detailed retinal and choroids structures. Between the retina 28 and the choroid 30 there is an outer segment of photoreceptor cells 32 including rods and cones, a subretinal space 34, and a layer of retinal pigment epithelium (RPE) 36. In a normal adult, retinal blood vessels 38, including capillaries, have walls or membranes 40 that contain no fenestrations or openings. In a normal adult, the large choroidal vessels 42 similarly have walls 44 that contain no fenestrations but the choriocapillaries 46 have walls that contain fenestrations 48. In an adult with AMD, there is either growth of new subretinal blood vessels whose walls or membranes are altered in that they also contain fenestrations, or the RPE cells are lost.

In exudative AMD, a lesion of subretinal neovascular tissue 52 develops in the choroid 30. The neovascular tissue 52 penetrates the RPE 36 and subretinal space 34, and extends into the area containing photoreceptor cells 32. The neovascular tissue 52 has membranes or walls 54 that are altered in having fenestrations 56 which permit fluid leakage into spaces surrounding photoreceptor cells 32, the subretinal space 34, and the RPE 36.

One type of AMD results in proliferation of new blood vessels in the subretinal area, typically the choroid. In the normal retina, both the large blood vessels and the capillaries have intact vessel walls. In the normal choroid, the large vessels have intact vessel walls, but the capillaries have fenestrations or openings in their walls. In patients with AMD, new blood vessels proliferate from the choriocapillaries through defects in Bruch's membrane beneath or on top of retinal pigment epithelium (RPE), and form vascular membranes. The resulting choroidal neovascularizations (new vessels in the choroid) occur in about 8-10% of all patients with AMD, and are also seen in patients with pathologic myopia and presumed ocular histoplasmosis syndrome, as well as other idiopathic conditions.

While the presence of the new vessels themselves is not problematic, any endogenous or exogenous fluid contained in these vessels (for example, blood, serous fluid, solubilized drug, etc.) will leak outside of the vessels and accumulate in the surrounding spaces. This accumulation of fluid can result in serous and hemorrhagic detachment of the RPE and neurosensory retina, and can lead to scarring in this area (fibrous deform scarring), resulting in decreased vision or even loss of vision. Thus, it is the fluid leakage from these new vessels in this type of AMD, called neovascular, exudative, or occult AMD, that is the cause of the resulting visual impairment. Therapies to prevent AMD are directed to slowing or stopping the formation or proliferation of new vessels in the choroid 30. Therapies to treat AMD are directed to at least partially damaging or destroy existing neovascular tissue 52, and/or interfering with its function. In either case, leakage of fluid from the new vessels is decreased, and the concomitant scarring and loss of vision is likewise diminished or eliminated. Another type of AMD occurs less commonly and is due to dead RPE cells; this is termed atrophic AMD. In either type of AMD, without treatment, many of the affected individuals will become legally blind.

Patients with early stage AMD can be diagnosed in an examination by the presence of drusen, an accumulation of dead outer segments of photoreceptor cells, under the RPE. Hyaline excrescences that are located in Bruch's membrane (lamina basalis choroidea) also form. The presence of large, soft drusen in the eye indicates a pre-stage of exudative AMD, and places patients at higher-than-average risk for developing neovascularizations, especially if one eye is already affected. To date, there are no known specific measures to prevent the occurrence of AMD. However, an anti-VEGF agent may have efficacy at an early stage of AMD (drusen), reducing or preventing its progression to full-fledged disease. Laser coagulation therapy results in drusen disappearance or reduction, but causes formation of scar tissue. The use of sunitinib maleate in the inventive method may preclude the need for such therapy and thus alleviate this problem. The use of sunitinib maleate in combination with PDT or laser coagulation may address this problem by ameliorating both AMD and resulting scar tissue.

For patients already diagnosed with AMD in one or both eyes, treatment involves targeting light (phototherapy) to the macular area to inhibit or impair the nascent defective blood vessels. For example, PDT uses a photosensitizing agent to locally and selectively destroy cells and/or tissues. The agent is administered into the vessels of a patient and transported to the retina. Immediately thereafter, or after an appropriate interval, the appropriate wavelength of light is directed to this specific area to activate the agent. Targeting low energy light to the area selectively activates the agent. The activated agent generates free radicals (e.g., singlet oxygen, hydroxyl radicals, other activated chemical species) that destabilize and destroy the new vessels in this area. For example, it damages the walls of the choriocapillaries and neovascular tissue, and leads to an initial vascular thrombus that may occlude the vessels.

PDT is generally directed to the lesion, but may also be administered to a generally circular area surrounding the lesion, up to about five disk diameters from the lesion. In one embodiment, PDT is administered to the lesion and an area about three to about five disk diameters from the lesion. In another embodiment, PDT is administered to the lesion and an area about one-half to about one disk diameter from the lesion. In still another embodiment, PDT is administered to the lesion. The size of the applied laser treatments may be in the range of about 1 mm to about 9 mm.

Selection of a photosensitive agent depends on the site(s) of tissue distribution requiring treatment, the mechanisms of action of the agents themselves, and their specific optimal absorption wavelengths. For example, tin ethyl etiopurpurin (SnET2) is frequently used as a photosensitive agent. SnET2 has lower persistence and severity of skin photosensitivity, it absorbs at longer wavelengths yielding better tissue penetration, it has a higher extinction coefficient resulting in increased potency and efficiency, ease of synthesis, and ability to be produced in a highly pure form. Protoporphyrin may be used as a photosensitizing agent. Protoporphyrin IX is a photoactive compound that is endogenously formed from 5-aminolevulinic acid (ALA) in the biosynthetic pathway of heme. ALA may be applied topically and is metabolized to protoporphyrin, the active photosensitizing agent. Laser irradiation is usually at a wavelength in the range of about 630 nm, or alternatively in the range of 670 nm. ALA may be administered orally in a bolus as an aqueous solution at a concentration of about 60 mg/kg body weight, or intravenously at a concentration of 30 mg/kg body weight. Other photosensitizing agents that may be used include, but are not limited to, benzoporphyrin derivative monoacid tube A (BPD-MA) and mono-l-aspartyl chlorine 6 (NPe6), with absorbance maxima in the range of about 660-690 nm, ATX-106, and indocyanine green (ICG). Verteporfin, a synthetic, chlorin-like porphyrin, may be intravenously injected at a dose of about 1-2 mg/kg, and activated by light at 50 J/cm² (absorbance peak of drug) from a non-thermal laser (for example, a diode laser) set at an intensity of 600 mW/cm² and a wavelength of 689 nm. Once activated, it generates singlet oxygen and other reactive oxygen radicals that selectively damage neovascular endothelial cells, and cause thrombus formation due to specific choroidal neovascular occlusion.

PDT has been reported to be of some benefit to patients having AMD. For example, one study (Arch. Ophthalmol 17:1329-1345, 1999) evaluated PDT in four hundred and two eyes from patients diagnosed with AMD in at least one eye. Treatment outcome was assessed by comparing the patient's ability to accurately read a conventional vision chart (one having about five letters per line) pre-treatment and post-treatment. At twelve months post-PDT, 61% of the eyes (246/402) lost fewer than 15 letters (that is, the patient lost less than about three lines on a standard visual chart), while 46% of the eyes (96/207) from patients undergoing treatment with a placebo lost fewer than 15 letters (p<0.001). At twenty-four months post-PDT, the visual acuity and contrast sensitivity was sustained in patients receiving PDT. A significantly greater percentage of these patients (58%) lost fewer than 15 letters, compared to patients undergoing treatment with a placebo (38%). However, only 16% of the patients receiving PDT had improved vision, compared to 7% of the patients receiving a placebo.

While PDT is used to treat patients with AMD, it has some drawbacks. One problem with PDT is that its effects are transient; patients receiving PDT must be retreated about every three months. Furthermore, the patients require at least five retreatments within the first two years merely to stabilize their condition, and before any therapeutic effect occurs. Additionally, these cumulative treatments damage the retina, further reducing the patient's visual acuity.

Ranibizumab has been administered by intravitreal injection in combination with verteporfin PDT to determine its effect on choroidal neovascularization. The combination caused a greater reduction in angiographic leakage than PDT only, as reported by Husain et al., April 2005 Arch Opthamol. 123:309, which is expressly incorporated by reference herein in its entirety. As previously described, ranibizumab is a derivative of the full-length antibody bevacizumab (Fab fragment), and is further modified to increase its affinity for VEGF.

In one embodiment, the method prevents, alleviates, or delays the onset of AMD in a patient by administering PDT simultaneously or concomitantly with sunitinib maleate. The method also prevents or delays the progression of AMD, and reduces further loss of vision in a patient having AMD, by administering PDT simultaneously or concomitantly with sunitinib maleate. The combination of PDT with sunitinib maleate enhances alleviation of AMD and/or its symptoms or sequelae. One skilled in the art will appreciate that enhanced alleviation encompasses any reduction in the duration, severity, type, etc. of the underlying pathology and/or its symptoms, and is not limited to complete efficacy, although therapeutic efficacy is included. The method thus encompasses preventing or delaying the onset of AMD, and/or maintaining visual acuity and preventing further loss of vision in patients with AMD. The method may generate free radicals and other activated chemical species that destabilize and destroy the new vessels via PDT, and reduce the formation of new blood vessels via anti-VEGF action of sunitinib maleate.

In addition to AMD, other ocular conditions may be treated with the inventive method and device. These conditions include, but are not limited to, blepharitis, conjunctivitis, keratitis, episcleritis, scleritis, papillitis (optic neuritis), uveitis, and/or endophthalmitis. The inventive method and device also may be used to reduce post-surgical inflammation.

It is reported that another anti-VEGF agent, bevacizumab, at a dose of 1 mg was administered as a single intravitreal injection to a patient with neovascular age-related macular degeneration. Rosenfeld et al. Ophthalmic Surg Lasers Imaging 2005; 36:331, which is expressly incorporated by reference herein in its entirety. There was resolution of subretinal fluid after one week, with improved macular appearance maintained for at least four weeks, and no observed inflammation.

In one embodiment of the method, sunitinib maleate is administered without PDT or any other type of phototherapy. Thus sunitinib maleate alone, or in combination with another agent, provides therapy without phototreatment. For example, while the inventive method may be used in conjunction with other therapies such as thermal laser coagulation, it may also be used without PDT, laser treatment, etc. but may include the addition of anti-proliferative agents, steroids, etc. administered with the inventive device or separately (e.g., subconjunctival depot steroid therapy), as known to one skilled in the art.

In another embodiment of the method, both PDT and sunitinib maleate are administered, but their administration is not restricted to a particular sequence. In one embodiment, PDT is administered and, essentially simultaneously with or immediately thereafter, sunitinib maleate is administered. In another embodiment, PDT is administered and sunitinib maleate is administered in the same treatment session, within a time frame of a few minutes or within a few hours. In another embodiment, PDT is administered and sunitinib maleate is administered after an interval from about one day up to about 90 days. In another embodiment, sunitinib maleate is administered and, essentially simultaneously with or immediately thereafter, PDT is administered. In another embodiment, sunitinib maleate is administered and PDT is administered in the same treatment session, within a time frame of a few minutes or within a few hours. In another embodiment, sunitinib maleate is administered and PDT is administered after an interval from about one day up to about 90 days. For embodiments in which PDT and sunitinib maleate are not administered essentially simultaneously, either may be administered first.

In one embodiment, after administering the photosensitive agent (verteporfin, protoporphyrin, SnET2, NPe6, ATX-106, ICG, etc.), the patient is treated using a laser to administer low energy levels of light at a wavelength appropriate to activate the photosensitive agent. Treatment with sunitinib maleate is then initiated essentially simultaneously or concomitantly. Essentially simultaneous treatment includes administration of both sunitinib maleate and low energy light within the same treatment session. Concomitant treatment includes administration either immediately thereafter or within a few hours, within 24 hours, or after an interval from about one day to ninety days.

In another embodiment, the patient is treated with sunitinib maleate, and is thereafter treated with PDT. The photosensitive agent may be administered either before or after sunitinib maleate treatment, depending upon a variety of factors such as the specific photosensitive agent used, the specific treatment protocol, etc. PDT is then essentially simultaneously or concomitantly initiated, as previously described. Factors such as patient comfort, tolerance to treatment, and convenience may be considered in selecting the appropriate treatment regime.

The combination of PDT and sunitinib maleate may provide synergistic benefits. One benefit is that the combined therapies induce regression of neovascular tissue. Besides patients with AMD, patients with diabetes who are particularly prone to proliferative retinopathy, a frequent cause of blindness, could benefit from this treatment. Another benefit is that the combined therapies do not produce additional neovascular tissue because of anti-angiogenic function. The combination of the two treatments thereby reduces the need for repetitive PDT treatments that damage the retina and further reduce the patient's visual acuity.

It should be understood that the embodiments of the present invention shown and described in the specification are only preferred embodiments of the inventor who is skilled in the art and are not limiting in any way. Therefore, various changes, modifications or alterations to these embodiments may be made or resorted to without departing from the spirit of the invention and the scope of the following claims.

What is claimed is:

1. A method comprising administering an anti-platelet derived growth factor (anti-pdgf) agent to an individual at: risk for post-surgical scar formation, the anti-PDGF agent administered at a dose sufficient to provide an anti-inflammatory effect to reduce post-surgical scar formation in the individual.

2. The method of claim 1 wherein the anti-PDGF agent is selected from the group consisting of imatinib, dasatinib, AMN107, and combinations thereof.

3. The method of claim 1 wherein the anti-PDGF agent is administered in combination with at least one anti-vascular endothelial growth factor (anti-VEGF) agent.

4. The method of claim 3 wherein the anti-VEGF agent is selected from the group consisting of bevacizumab, ranibizumab, pegaptanib, sunitinib maleate, TNP470, integrin av antagonists, 2-methoxyestradiol, paclitaxel, P38 mitogen activated protein kinase inhibitors, anti-VEGF siRNA, and combinations thereof.

5. The method of claim 1 wherein the anti-PDGF agent is administered prophylactically.

6. The method of claim 1 wherein the individual is at risk for restenosis.

7. The method of claim 1 wherein the anti-PDGF agent is administered ocularly.

8. The method of claim 1 wherein the anti-PDGF agent is administered systemically.

9. Use of an anti-platelet-derived growth factor (anti-PDGF) agent in a biocompatible composition with at least one excipient for anti-inflammatory effect to reduce scar formation in an individual undergoing a surgical procedure.

10. The use of claim 9 wherein the individual is undergoing a cardiac surgical procedure.

11. The use of claim 9 wherein the individual is undergoing angioplasty.

12. The use of claim 9 wherein the individual is undergoing stent insertion.

13. The use of claim 9 wherein the individual is undergoing a vascular graft.

14. The use of claim 9 wherein the anti-PDGF agent is imatinib.

15. The use of claim 9 wherein the anti-PDGF agent is dasatinib.

16. The use of claim 9 wherein the anti-PDGF agent is AMN107.
